(19) United States
(12) Patent Application Publication
(10) Pub. No.: US 2003/0181904 A1
(43) Pub. Date: Sep. 25, 2003

(54) Electrosurgical cutting, coagulating and suction instrument

(76) Inventors: Andy H. Levine, Newton, MA (US); John Cvinar, Winchester, MA (US); Marc Joshua Tolkoff, Brookline, MA (US); Scott Naisbitt, St Louis, MO (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/348,703

(22) Filed: Jan. 22, 2003

Related U.S. Application Data
(60) Provisional application No. 60/349,933, filed on Jan. 23, 2002.

(51) Int. Cl. A61B 18/14

(52) U.S. Cl. 606/45; 606/49; 604/35

(57) ABSTRACT

An electrosurgical instrument that combines cutting, coagulating and suctioning functionality in a single hand-held instrument. The instrument provides a combination suction-coagulation device that can selectively deliver electrical energy through either a blunt suction coagulation end or a “sharp” needle or blade end and a convenient means for a surgeon to alternate between these two modalities. A cooling medium in a sealed cavity may be incorporated either (i) as a heat-pipe within one side of electrode wall around the suction lumen, (ii) as a second coaxial lumen within the circumscribing electrode wall, (iii) in an adjacent but thermally coupled heat-sink pipe attached either external or internal to (but not fully occluding) the suction lumen, or (iv) in a heat-pipe far proximal to the electrode ring but thermally coupled.

### CROSS REFERENCE TO RELATED APPLICATIONS

The present application cross-references and incorporates by reference and claims the benefit of U.S. provisional patent application serial No. 60/349,933, filed on Jan. 23, 2002.

### FIELD OF THE INVENTION

The present invention relates in general to electrosurgical devices, more particularly, to an electrosurgical instrument that combines suction capability with cutting and coagulating electrodes and the ability to switch between the cutting and coagulating electrodes during surgical procedures, especially endoscopic procedures.

### BACKGROUND OF THE INVENTION

Hemostasis during surgical procedures is commonly achieved using electrosurgery. Electrosurgical devices are typically handheld and insulated except at the working tip where high frequency electrical energy is delivered through a conductive element at the surgical site. If cutting of tissue is desired, a tip with a “sharp” or electrically focusing edge is desired. If coagulation of tissue is desired, a blunt or electrically spreading electrode is desired. In addition, the tissue to be cauterized is often obscured by a pool of blood or smoke.

Consequently, a significant need exists for a versatile electrosurgical instrument that combines both cutting and coagulation and further provides a source of vacuum at the tissue site during coagulation to clear the field of blood prior to and during the application of the electrical energy.

### BRIEF SUMMARY OF THE INVENTION

The present invention offers an improved electrosurgical instrument that combines cutting, coagulating and suctioning functionality in a single hand-held instrument.

One objective of the present invention is to provide a combination suction-coagulation device that can selectively deliver electrical energy through either a blunt suction coagulation end or a “sharp” needle or blade end. It is a further objective to provide convenient means for a surgeon to alternate between these two modalities.

Several distinct embodiments meet these objectives. In one embodiment, the apparatus contains two independent electrodes, a “sharp” electrosurgery needle or blade for cutting and a blunt electrode for coagulating. The blunt, electrode may be comprised of an insulated conductive tube with an exposed distal tip for delivery of electrical energy and a hollow axial lumen through which suction may be applied. Either electrode may be extended distally or retracted proximally with respect to the other electrode. This extension or retraction may be accomplished by any of several mechanisms including a manual slider coupled to the cutting blade or blunt suction component or both, a gear or gears coupling manual rotation of a dial knob to movement of one component along the other, a combination of both of these, or a releasing mechanism that allows manual sliding of the tips. Electrical energy may be allowed to pass only through the distally deployed electrode by controlled electrical switching or insulation. The surgeon may thus safely apply blunt coagulation while suctioning, or alternatively retract the suction and deploy the cutting blade.

In another embodiment, two distinct electrode components, the sharp electrode or the blunt suction coagulation ring electrode, may be alternately exchanged and attached by insertion into a single handle, as desired by the surgeon.

In another embodiment, an electrosurgery device with an integral blade electrode may be constructed along with a separate attachable blunt suction coagulation ring extension. This extension may be designed so that it sheaths the electrosurgery blade and extends the suction lumen and electrical energy to the operative field. The attachment may be reversibly removed and replaced as desired by the surgeon.

In an additional embodiment, an integrated apparatus may be constructed with the sharp electrode protruding from one end of the handle and the blunt suction coagulation ring electrode protruding from the other. The handle may then be reversibly oriented as desired by the surgeon to bring either electrode into contact with the operative field. Suction may be applied to either or both protruded electrode ends.

It is a further objective of the present invention to provide a means for maintaining cool electrode tips in each embodiment. Each electrode in the above embodiments may incorporate a temperature control component such as a heat pipe, as described in U.S. Pat. Nos. 5,647,871, 6,074,389, and 6,206,876, and all of which are incorporated by reference herein in their entirely. Alternatively, the combination device may have one electrode constructed of material with high thermal conductivity that is thermally coupled to the other electrode that includes an active cooling means. For example, the blunt suction coagulation ring electrode might be constructed of copper and conduct heat to a retracted or sheathed sharp electrode heat pipe. Alternatively, the blunt suction coagulation component may itself contain a heat pipe.

It is a further objective of the present invention to provide a means for the electrode shafts to be easily bendable as desired by the surgeon to improve access to the surgical site.

It is a further objective of the present invention to provide for smoke evacuation during needle or blade electrosurgery. Aspiration of smoke may occur through the blunt suction component lumen even when the blunt coagulation electrode tip is retracted, reversed or removed.

It is a further objective of the present invention to be comfortably hand-held, ergonomic, and similar in size to standard instruments.

The present invention provides a means for combining electrode tip temperature control technology with both blade and blunt suction cautery in a single instrument. This has two major advantages over existing art. First, the low tip temperature results in improved performance: more effective cutting and coagulation, decreased smoke and char, better field visualization and an expected decrease in thermal injury and postoperative pain. Second, the single combination device replaces two existing instruments.

The suction shaft may alternatively be a non-conductive tube that may slide adjacent to or over a cooled needle or blade electrode in order to locally aspirate at the site of blade.

It is a separate objective to provide a mechanism for maintaining a cool tip in a simple non-combination blunt suction coagulation device. This cooled suction cautery device may take the standard form of a single common shaft affecting both suction and coagulation, in which the insulated tubular electrode has an exposed ring tip for delivery of electrical energy and a hollow axial lumen through which vacuum suction may be applied. A cooling medium in a sealed cavity may be incorporated either (i) as a heat-pipe within one side of electrode wall around the suction lumen, (ii) as a second coaxial lumen within the circumscribing electrode wall, (iii) in an adjacent but thermally coupled heat-sink pipe attached either external or internal to (but not fully occluding) the suction lumen, or (iv) in a heat-pipe far proximal to the electrode ring but thermally coupled (i.e., via a copper electrode attached to a heat-pipe within the handle).

The present invention has, without limitation, application in conventional endoscopic and open surgical instrumentation as well as application in robotic-assisted surgery. The application is also useful in monopolar or bipolar electrosurgical applications.

### BRIEF DESCRIPTION OF THE FIGURES

These and other features, aspects, and advantages of the invention will become more readily apparent with reference to the following detailed description of a presently preferred, but nonetheless illustrative, embodiment when read in conjunction with the accompanying drawings. The drawings referred to herein will be understood as not being drawn to scale, except if specifically noted, the emphasis instead being placed upon illustrating the principles of the invention. In the accompanying drawings:

FIG. 1 is a perspective view of one embodiment of the invention wherein two separate removable electrodes, a blunt suction ring and a sharp tip or blade electrode may be interchangeably inserted into a single handle;

FIG. 2 is a perspective view of an alternate embodiment wherein a blunt suction coagulation ring electrode extension may reversibly sheath a blade electrode integral to the device handle;

FIGS. 3A and B are perspective views of an alternate configuration of the embodiment of FIG. 2 and having a sliding mechanism for alternating between blade and blunt suction cautery electrodes;

FIGS. 4A and B are prespective views of an alternate configuration of the embodiment of FIG. 2 and having a sliding mechanism for alternating between blade and blunt suction cautery electrodes;

FIGS. 5A and B are prespective views of an alternate configuration of the embodiment of FIG. 2 and having a sliding mechanism for alternating between blade and blunt suction cautery electrodes;

FIGS. 6A and B are prespective views of an alternate configuration of the embodiment of FIG. 2 and having a sliding mechanism for alternating between blade and blunt suction cautery electrodes;

FIGS. 7a to I illustrate embodiments of an electrosurgical blunt suction coagulation device incorporating means for maintaining a cooled tip.

FIG. 8 is a schematic illustration of a reversible working end of a electrocautery device; and

FIGS. 9A and B schematically illustrate an alternate embodiment of a dual tip electrocautery device where the insulation and suction mechanisms slide; and

FIG. 10 is a schematic illustration of an electrocautery device having a blunt electrode and sharp blade electrode offset from each other.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is understood that any one or more of the following-described embodiments, expressions of embodiments, examples, methods, etc. can be combined with any one or more of the other following-described embodiments, expressions of embodiments, examples, methods, etc. In general, the present invention relates to the end-effector or working end of an electrosurgical instrument integrating means for both sharp and blunt suction electrocautery and a mechanism for alternating between the two. The invention is suitable for use in multiple surgical instruments, such as those disclosed in U.S. Pat. Nos. 5,599,350 and 5,709,680, and modified accordingly to fit the current invention as is readily known to one skilled in the electrosurgical instrument art. Therefore, the description of the invention does not include the surgeon interface, which can take multiple configurations and easily designed by one skilled in the art.

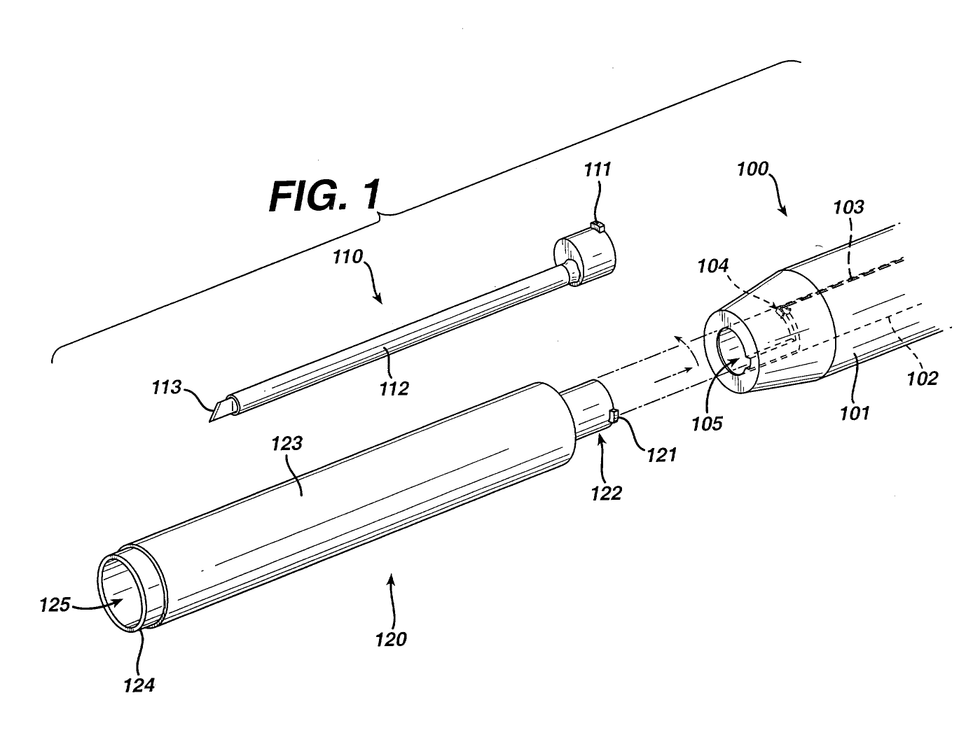

An embodiment of the invention is shown in FIG. 1. In this embodiment, separate electrode tips—an electrocautery blade or a blunt suction coagulator ring—may be interchangeably inserted into a single handle as desired by the surgeon. The handle 100 has a housing 101 sized to be comfortable in a hand. It contains a conventional lumen 102 and wiring 103 that is connected proximally (not illustrated) by standard means to a conventional vacuum source and radiofrequency (“RF”) generator (either monopolar or bipolar), respectively. At the distal end of the handle, there are attachment sites 104 and 105 into which blade electrode 110 or blunt suction cautery electrode 120 are inserted.

The needle or blade electrode 110 has an adapter 111 for insertion into the handle attachment site 104, where electrical contact is made via wire 103 to a power source. The blade electrode 110 is surrounded by insulation 112 except at the distal tip 113, which is exposed for delivery of electrical energy to the operative field. This electrode 110 may also contain a cavity containing a cooling medium (not illustrated) as previously taught in order to maintain a low tip temperature during electrocautery. When the blade electrode is attached to the handle, the handle suction lumen 105 remains exposed to aspirate smoke.

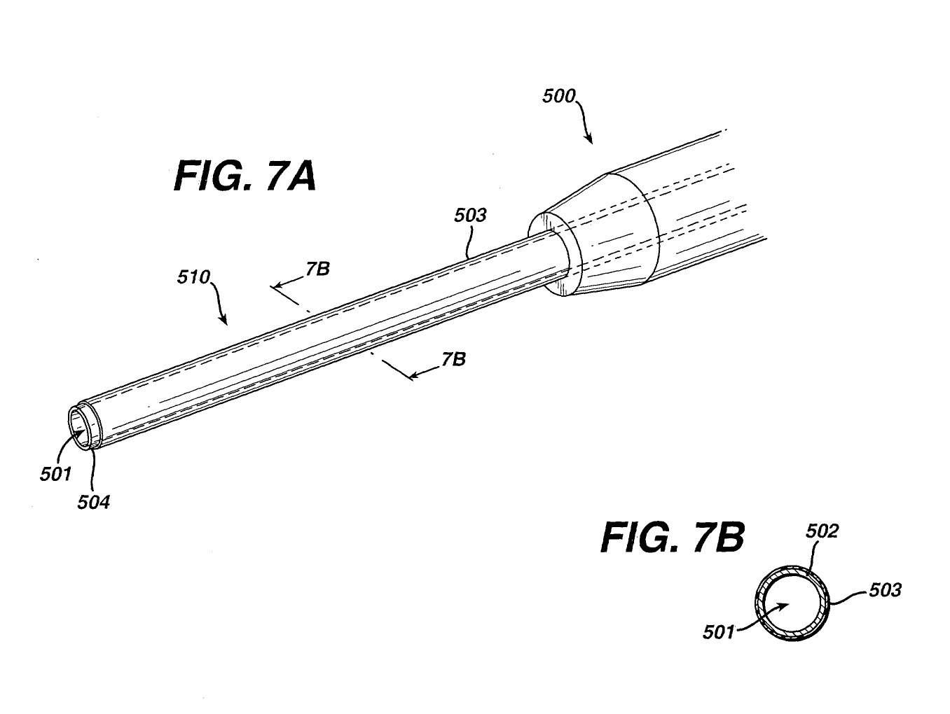

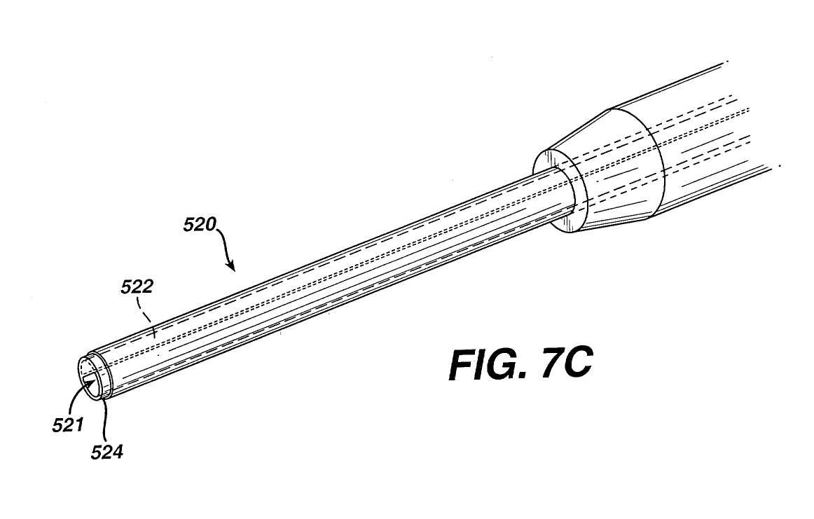

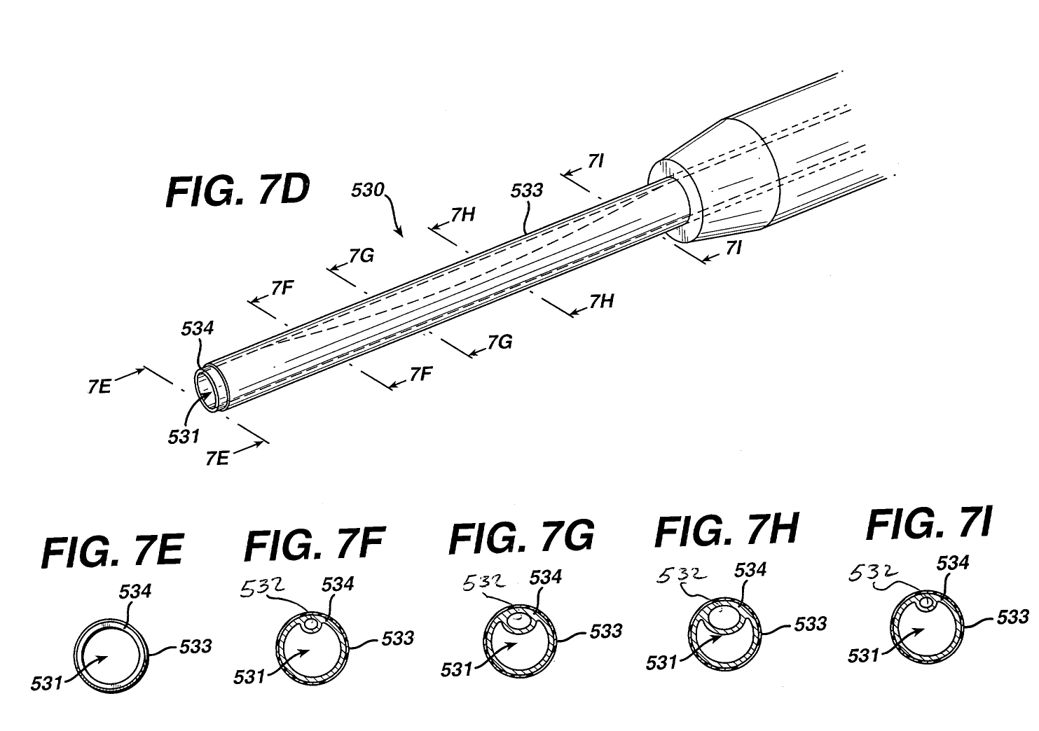

As desired by the surgeon, the sharp electrode 110 may be removed and the blunt suction-cautery electrode 120 may be inserted in its place. This blunt suction electrode 120 has an adapter 121 for insertion into the handle attachment site 104, where electrical contact is made via wire 103 to a power source. The blunt suction electrode 120 also has a circumferential step 122 in the proximal end for firm insertion into the handle suction lumen 105. This provides solid attachment of the electrode 120 and leak-free extension of the vacuum suction lumen from the handle 105 to the distal end 125 of the electrode 120, which comes in contact with the operative field. The electrode is surrounded circumferentially with insulation 123 throughout its length except at the adapter 121 and the blunt ring tip 124, which is exposed for blunt delivery of electrical energy to the patient. A low tip temperature is maintained by constructing the blunt suction cautery electrode 120 from a material having high thermal conductivity such as copper, or by incorporating a cavity containing a cooling medium as detailed in FIG. 7 below. Blunt coagulation via exposed electrode ring 124 and suction via lumen 125 may be performed simultaneously. Preferably, electrodes 110 and 120 are made from a malleable material so the surgeon may bend either electrode for operative convenience.

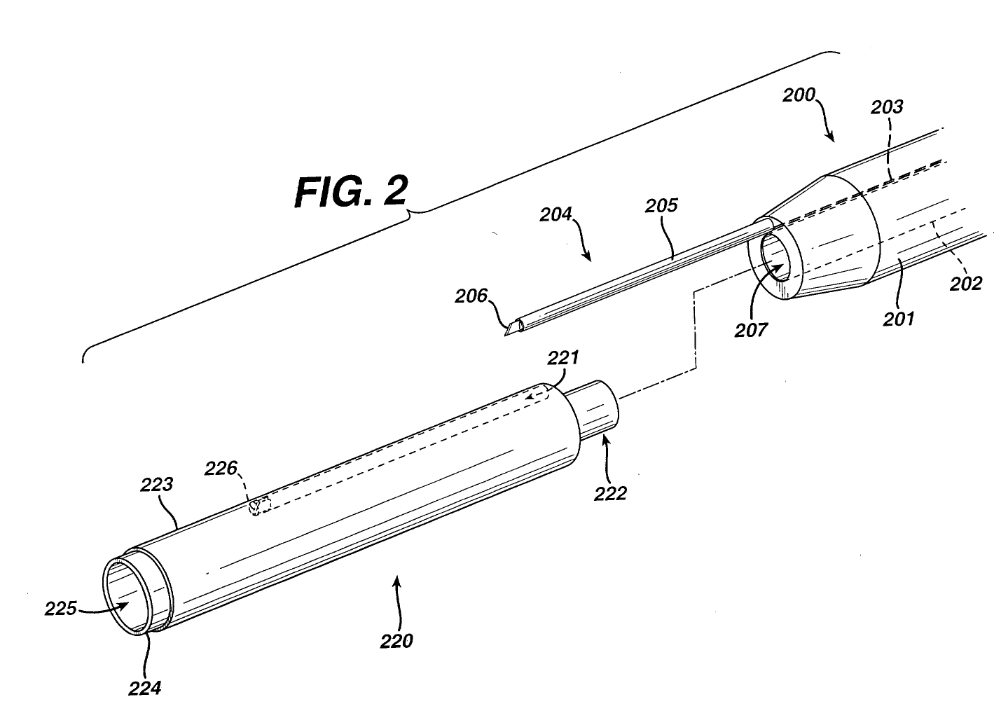

FIG. 2 illustrates a second embodiment of the invention. In this embodiment, a blunt suction cautery extension is removably attached over a sharp electrode that is integrated into the handle. The handle 200 contains housing 201, suction lumen 202 and wiring 203 all having conventional attachments proximally to standard equipment as described above and in prior art. Integral with the handle is the blade or needle electrode 204, which is coupled proximally via wiring 203 to the conventional power source. The electrode 204 is surrounded circumferentially by insulation 205 except at the tip 206, which is exposed for delivery of electrical energy to the operative field or for electrical coupling of the blunt suction cautery extension 220. The blade electrode 204 may contain a cavity containing a cooling medium (not illustrated) as previously taught in order to maintain a low tip temperature during electrocautery. During sharp electrocautery, the exposed handle suction lumen 207 may function for smoke evacuation.

Should the surgeon desire blunt suction-coagulation, the electrode extension 220 may be attached. The wall of the extension 220 contains a hollow pocket 221 that may conveniently sheath the blade electrode 204. The exposed tip 206 of the blade electrode 204 makes a solid electrical connection 226 with the blunt electrode extension 220, safely and securely electrically coupling the extension 220 and cautery ring 224 to the wire 203 and power source. The extension also has a radial step 222 at its proximal end for firm insertion into the handle lumen 207. This provides for secure attachment of the extension 220 and leak-free extension of the vacuum suction lumen from the handle 207 to the distal end 225 which will come in contact with the operative field. The electrode extension 220 is surrounded circumferentially with insulation 223 throughout its length except at the blunt ring tip 224, which is exposed for delivery of electrical energy to the patient.

Maintaining a low tip temperature is particularly convenient in this embodiment, since if the blade electrode 204 incorporates an internal cavity with a cooling medium, it is thermally coupled to the suction cautery extension 220, obviating the need for additional means of cooling. Blunt coagulation via exposed electrode 224 and suction via lumen 225 may be performed simultaneously as desired. The surgeon may bend either the sharp electrode 204 or extension 220 as desired, and may remove or replace the extension 220 as needed for blunt suction or sharp blade electrocautery.

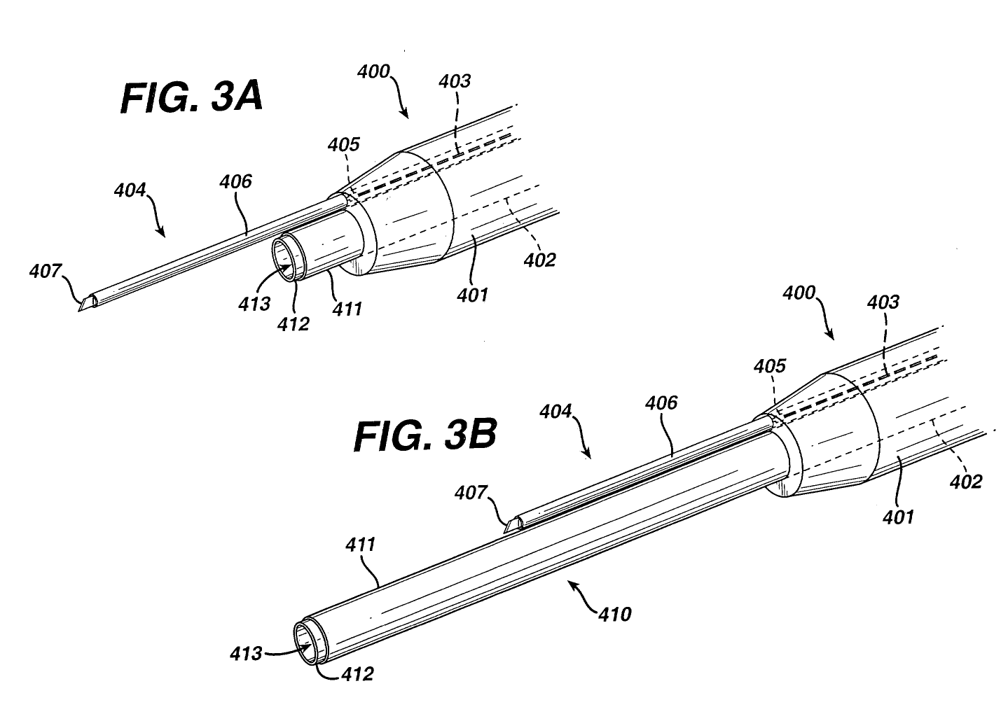

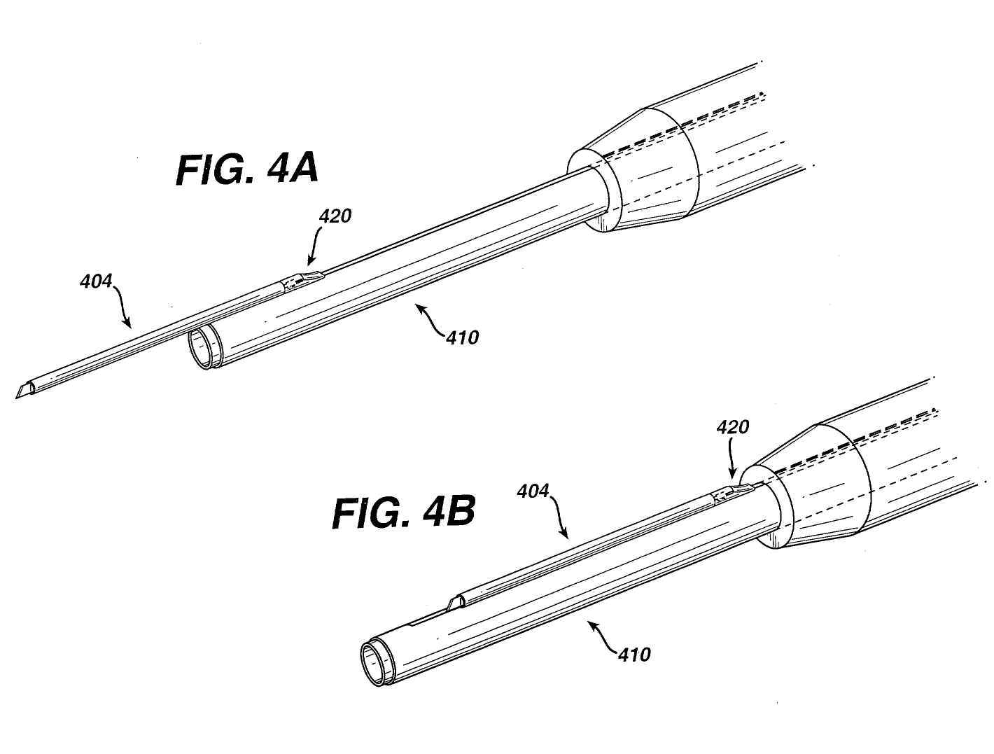

FIGS. 3 and 4 illustrate alternate configureations of the invention wherein a sliding mechanism is used to alternate between blade and blunt suction coagulation electrodes. In FIGS. 3A and B, the suction cautery electrode and suction tubing slide with respect to a fixed handle and integral blade electrode. The handle 400 has housing 401, suction lumen 402, and wiring 403 that may connect proximally by standard means to a conventional vacuum suction and electrical power source (not shown). Significantly, the suction electrode shaft is adapted so that it can slide within the handle by utilizing any of several independent mechanisms such as a geared dial, an external slider, or a release button (not shown). Distally, the blade or needle electrode 404 may be identical to electrode 204 described in FIG. 2. The electrode 404 makes an electrical connection 405 to wiring 403, is surrounded by insulation 406 except at the tip 407, which is exposed for delivery of electrical energy to the patient.

The blunt suction cautery electrode 410 contains a lumen 413 that is continuous with the suction lumen 402 within the handle 400. It is surrounded circumferentially by insulation 411 except at the ring tip 412, which is exposed for delivery of blunt electrocautery. An electrical switching mechanism (not shown) may be incorporated so that current flows through the blade electrode only when the blunt suction electrode is fully retracted, and through the blunt suction ring electrode only when it is fully extended, in order to protect against inadvertent burns.

In FIGS. 4A and B, the blade or needle cautery electrode 404 may slide with respect to a fixed handle and integral suction cautery electrode 410. This device is quite similar to that of FIG. 3, except that the blade electrode 404 may retract or extend over the suction coagulator electrode 410. This may be accomplished using a manual slider 420 that moves along a track 421. Again, an electrical switching mechanism (not shown) may be incorporated to ensure current passes only through the distally deployed electrode.

Alternatively, the blade electrode 404 may slide as drawn in FIG. 4 while the suction coagulation ring electrode and shaft 410 may also slide within the handle as drawn in FIG. 3. This allows for simultaneous retraction of the suction ring electrode and extension of blade electrode. This combination provides enough length variation so that the blade and blunt suction coagulator may be operated at lengths identical to conventional stand-alone instruments.

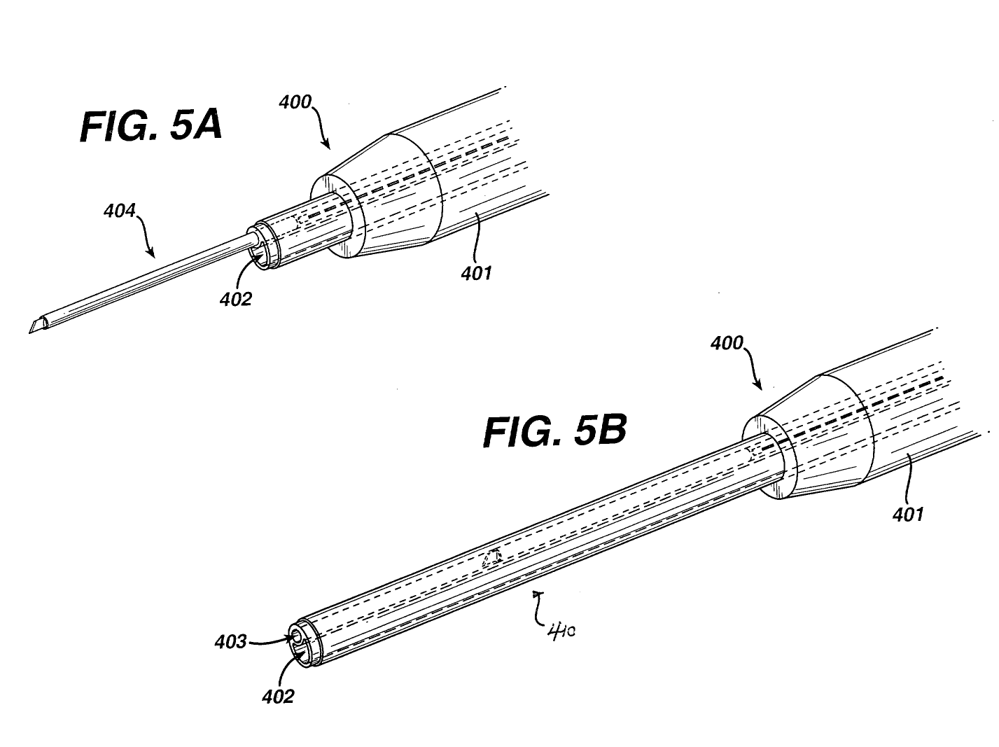

Referring now to FIGS. 5A and B, a sliding blade electrode 404 is incorporated within the wall of the sliding blunt suction coagulation electrode 410 rather than external and adjacent to it. In this embodiment, creation of a second lumen 403 within which the blade electrode 404 may slide may result in wall bulging that partially infringes upon but not occlude suction lumen 402. Again, the device embodied herein may be designed such that either one or both of the electrodes may slide with respect to the other by various mechanisms such as a geared dial, slider, or manual release.

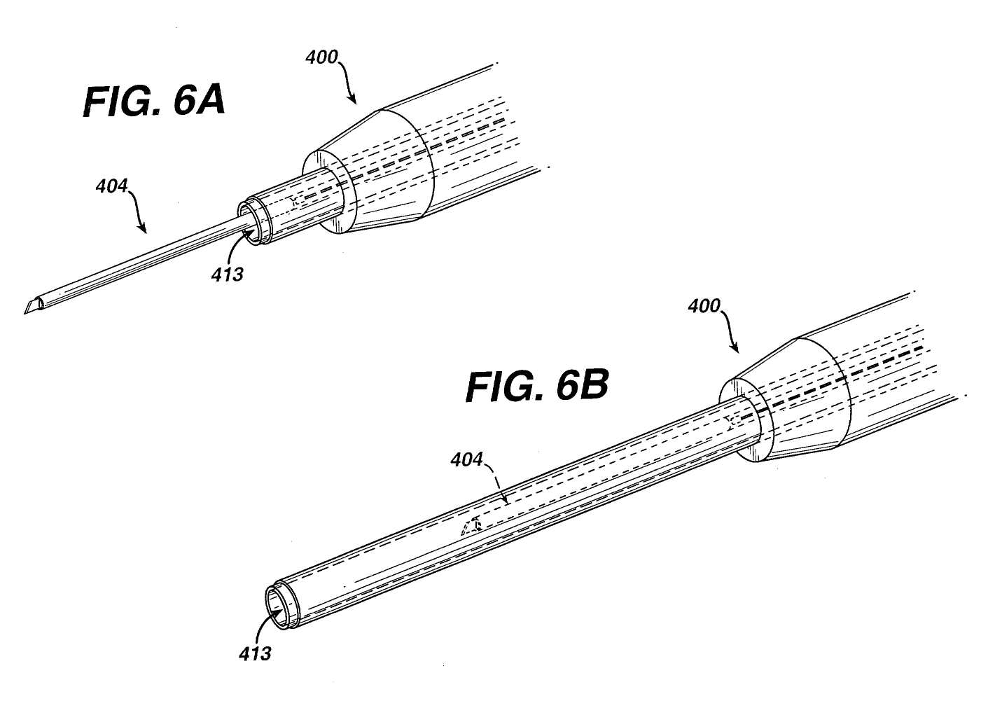

In FIGS. 6A and B, an alternate configuration is illustrated wherein the sliding blade electrode 404 is placed within the suction lumen 413 of the sliding blunt suction coagulation electrode. Again, the device may be designed so that either or both electrodes may slide with respect to the other by any of several mechanisms. The suction shaft may also be non-conductive tubing and slide adjacent to or over a needle or blade electrode as desired for simple aspiration at the site of blade electrocautery.

FIGS. 7A to I illustrate various embodiments of a non-combination blunt suction coagulation device that maintains a cool electrode tip by incorporating a cooling medium in a sealed cavity. In FIG. 7A an embodiment is illustrated in a conventional form comprising a handle 500 indistinguishable from existing art and a single common shaft 510 that affects both suction and coagulation. The tubular electrode is surrounded by insulation 503 and has an exposed blunt ring tip 504 for delivery of electrical energy to the operative field and a hollow axial lumen 501 through which vacuum suction may be applied. In this embodiment, the cooling medium is incorporated within a sealed coaxial cavity 502 within the circumscribing electrode wall as shown in FIG. 7B. Alternatively, as shown in FIG. 7C the cooling medium is in a sealed cavity incorporated as a heat-sink pipe 522 within one side of electrode wall surrounding the suction lumen 521.

FIGS. 7D and 7E through 7I illustrate a modified heat sink pipe 532 having a variable cross section along shaft 533. This has the advantage of maintaining the distal suction lumen 531 and functional coagulation ring 534 fully patent and symmetric, like a conventional non-cooled instrument.

Alternative embodiments (not shown) may incorporate the cooling medium in an adjacent but thermally coupled heat-sink pipe attached either external or internal to (but not fully occluding) the suction lumen or far proximal but thermally coupled to the electrode ring—such as a copper electrode attached to a heat-pipe within the handle.

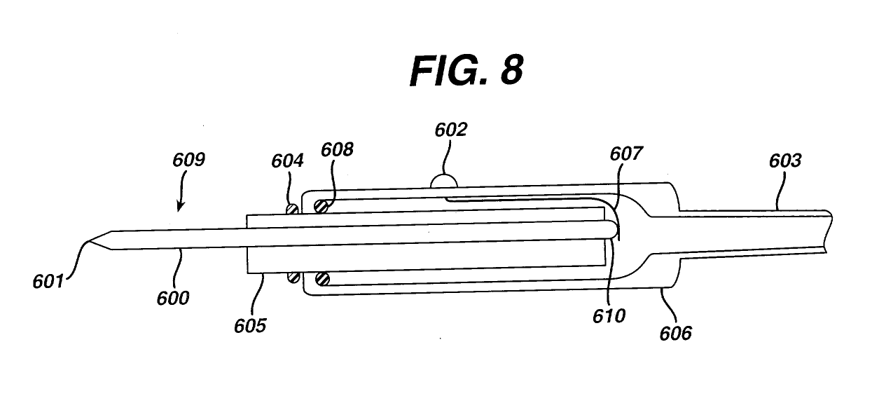

FIG. 8 shows a reversible tip design that allows the physician to have either the sharp end 601 or the blunt, suction end 610 exposed depending on the surgical need. As shown, the sharp tip 601 is exposed and the suction-blunt tip 607 is hidden inside the handle 606. The o-ring 608 seals the body from the suction applied by the hose 603. This permits the suction opening 605 to be exposed to pull smoke from the operative field. Button 602 is an electrical switch that closes the circuit of the RF generator (not shown). Electrical contact 607 is in contact with the electrode 600. The stop 604 sets the depth that the tip assembly 609 engages with the handle. The tip 609 can be withdrawn by the surgeon, turned around and then placed back into the handle 606 to now expose the blunt suction end 610 and hide the sharp end 601. The electrical contact 607 is springy and therefore retracts to meet the shorter end that is engaged in the handle to contact the sharp tip 601 in this position.

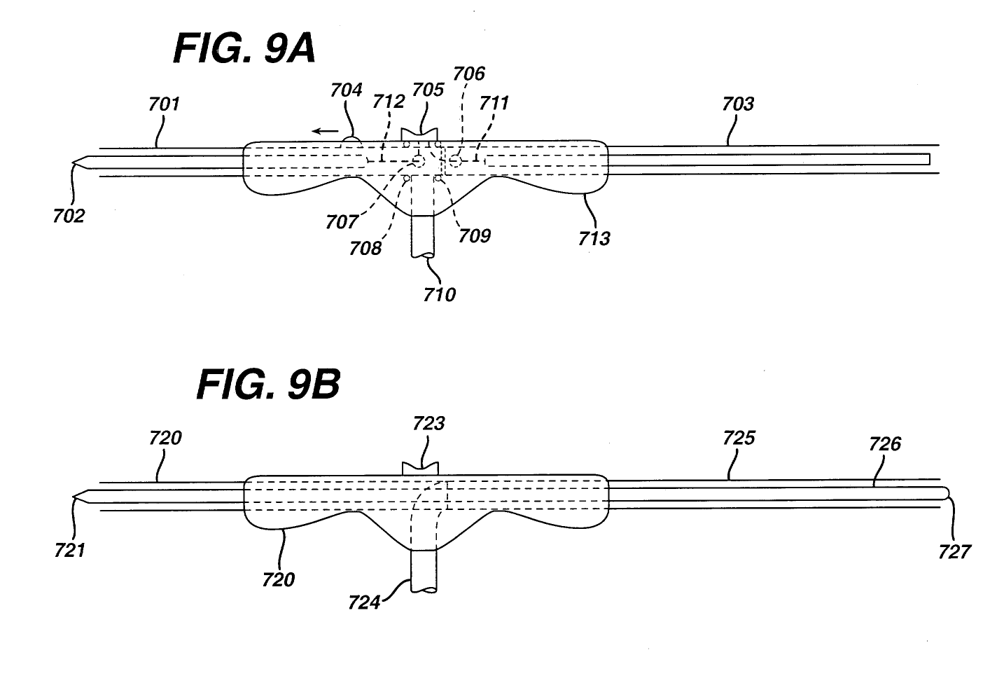

FIG. 9A shows a dual tip design Where the insulation/suction tube 701 and 703 may be moved using switch 704 to cover the tip that is not in use. This is important to reduce the likelihood of inadvertent burns. By also moving the suction tubes, the suction is only active on, the tip being used. When the switch 704 is moved towards the sharp tip 702, the tube 701 and 703 also moves and the holes 707 and 706 move to expose one hole 706 to the suction tube 710. O-rings 708 and 709 provide a seal. In this way, the sharp end 702 is covered with the insulating tube 701 and the suction is applied to the blunt end 703. The electrical switch 705 activates either the sharp end or the blunt end depending on which direction it is depressed.

FIG. 9B shows a dual tip design with the sharp end exposed at one end 721 and the blunt, suction end exposed at the other 727. The suction 724 is attached to the blunt end and is always on.

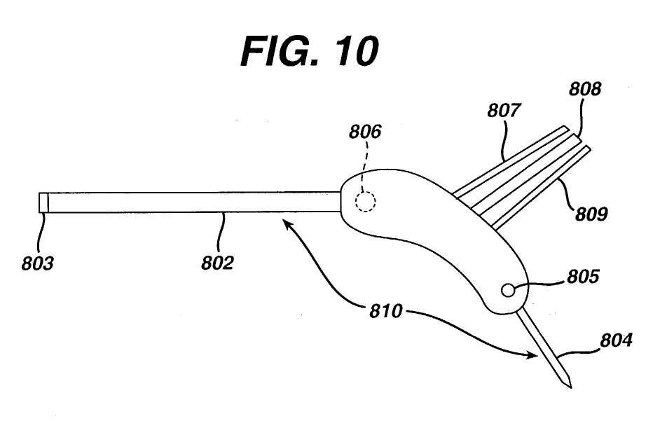

FIG. 10 shows a dual tip design with the sharp electrode 804 and the blunt electrode 803 at an angle 810 from each other. This permits the physician to use one end while pointing the other end away from the user. Electrical buttons 806 and 805 are on opposite sides of the handle 801. Angle 810 may most likely be between 90 and 150 degrees. The electrical and suction connections 807, 808 and 809 exit the handle at the mid point of the handle.

While the present invention has been illustrated by description of several embodiments, it is not the intention of the applicant to restrict or limit the spirit and scope of the appended claims to such detail. Numerous variations, changes, and substitutions will occur to those skilled in the art without departing from the scope of the invention. Moreover, the structure of each element associated with the present invention can be alternatively described as a means for providing the function performed by the element. Accordingly, it is intended that the invention be limited only by the spirit and scope of the appended claims.

We claim:

1. An electrosurgical device capable of receiving a radiofrequency energy source therein comprising:
a shaft having a distal end and a longitudinal axis, a lumen disposed along the longitudinal axis terminating at the distal end and coupled to a vacuum source and the shaft adapted to receive radiofrequency energy;
a first electrode adapted for performing a first electrosurgical procedure and releasably attachable to the distal end and electrically coupled to the radiofrequency energy source; and
a second electrode adapted for performing a second electrosurgical procedure and releasably attachable to the distal end and electrically coupled to the radiofrequency energy source.

2. The electrosurgical device of claim 1, wherein the first electrode provides for tissue cutting.

3. The electrosurgical device of claim 1, wherein the second electrode provides for tissue coagulation.

4. The electrosurgical device of claim 1, wherein the shaft further comprises an internal cavity in which a cooling medium is contained.

5. The electrosurgical device of claim 1, wherein at least one of the first and second electrodes are malleable.
